(19) United States
(12) Patent Application Publication
(10) Pub. No.: US 2026/0004892 A1
(43) Pub. Date: Jan. 1, 2026

(54) PHARMACOGENOMICS INDUCED PROTEIN FUNCTION OF THERAPEUTIC TARGETS

(71) Applicant: International Business Machines Corporation, Armonk, NY (US)

(72) Inventors: ARITRA BOSE, Astoria, NY (US); FILIPPO UTRO, Pleasantville, NY (US); Laxmi Parida, Mohegan Lake, NY (US)

(21) Appl. No.: 18/755,017

(22) Filed: Jun. 26, 2024

(51) Int. Cl. G16C 20/50; G16B 30/00; G16B 40/00

(52) U.S. Cl. CPC G16C 20/50; G16B 30/00; G16B 40/00

(57) ABSTRACT

A set of candidate drugs is selected based on one or more outcomes related to one or more diseases and variants linked with the selected set of candidate drugs are obtained. One or more protein sequences related to the selected set of candidate drugs are collated. Pairs of variants and protein sequences are generated for each drug of the set of candidate drugs. A contrastive learning model is trained using the generated pairs of variants and protein sequences and a downstream task is performed using the contrastive learning model.

### BACKGROUND

The present invention relates generally to the electrical, electronic and computer arts and, more particularly, to machine learning and pharmaceuticals.

Pharmacogenomics (PGx) studies the role of genes and genetic variants in responses to medications. Proteomics describes a protein structure/sequence. Through gene expression, the genetic variants are linked to protein function (for example, particular genes may trigger certain proteins). Germline genetic variants, such as Single Nucleotide Polymorphisms (SNPs), have been shown to significantly associate with complex diseases and can exacerbate diseases, cause disease resistance or drug resistance, and the like. SNPs are often categorized as missense, pathogenic, deleterious or disease-causing as they are found to alter protein function.

### BRIEF SUMMARY

Principles of the invention provide systems and techniques for pharmacogenomics induced protein function of therapeutic targets. In one aspect, an exemplary method includes the operations of selecting a set of candidate drugs based on one or more outcomes related to one or more diseases; obtaining variants linked with the selected set of candidate drugs; collating one or more protein sequences related to the selected set of candidate drugs; generating pairs of variants and protein sequences for each drug of the set of candidate drugs; training a contrastive learning model using the generated pairs of variants and protein sequences; and performing a downstream task using the contrastive learning model.

In one aspect, a computer program product comprises one or more tangible computer-readable storage media and program instructions stored on at least one of the one or more tangible computer-readable storage media, the program instructions executable by a processor, the program instructions comprising selecting a set of candidate drugs based on one or more outcomes related to one or more diseases; obtaining variants linked with the selected set of candidate drugs; collating one or more protein sequences related to the selected set of candidate drugs; generating pairs of variants and protein sequences for each drug of the set of candidate drugs; training a contrastive learning model using the generated pairs of variants and protein sequences; and performing a downstream task using the contrastive learning model.

In one aspect, a system comprises a memory and at least one processor, coupled to the memory, and operative to perform operations comprising selecting a set of candidate drugs based on one or more outcomes related to one or more diseases; obtaining variants linked with the selected set of candidate drugs; collating one or more protein sequences related to the selected set of candidate drugs; generating pairs of variants and protein sequences for each drug of the set of candidate drugs; training a contrastive learning model using the generated pairs of variants and protein sequences; and performing a downstream task using the contrastive learning model.

In one aspect, a system comprises a first transformer configured to generate embeddings for a contrastive learning model based on variants, the variants linked with a selected set of candidate drugs, the set of candidate drugs selected based on one or more outcomes related to one or more diseases; a second transformer configured to generate embeddings for the contrastive learning model based on protein sequences; a contrastive learning model trained using the variant-based embeddings, the protein sequences-based embeddings and generated pairs of the variants and the protein sequences; and a software component configured to implement a downstream task using the contrastive learning model.

As used herein, “facilitating” an action includes performing the action, making the action casier, helping to carry the action out, or causing the action to be performed. Thus, by way of example and not limitation, instructions executing on a processor might facilitate an action carried out by instructions executing on a remote processor, instructions that control pharmaceutical synthesis equipment, or the like, by sending appropriate data or commands to cause or aid the action to be performed. Where an actor facilitates an action by other than performing the action, the action is nevertheless performed by some entity or combination of entities.

Techniques as disclosed herein can provide substantial beneficial technical effects, as will be discussed further below. Features and advantages will become apparent from the following detailed description of illustrative embodiments thereof, which is to be read in connection with the accompanying drawings.

### BRIEF DESCRIPTION OF THE DRAWINGS

The following drawings are presented by way of example only and without limitation, wherein like reference numerals (when used) indicate corresponding elements throughout the several views, and wherein:

FIG. 1 is a high-level workflow for learning associations between proteins and variants (such as SNPs), in accordance with an example embodiment;

FIG. 2 is a flowchart for an example method for learning associations between proteins and SNPs, in accordance with an example embodiment; and

FIG. 3 depicts a computing environment according to an embodiment of the present invention.

It is to be appreciated that elements in the figures are illustrated for simplicity and clarity. Common but well-understood elements that may be useful or necessary in a commercially feasible embodiment may not be shown in order to facilitate a less hindered view of the illustrated embodiments.

### DETAILED DESCRIPTION

Principles of inventions described herein will be in the context of illustrative embodiments. Moreover, it will become apparent to those skilled in the art given the teachings herein that numerous modifications can be made to the embodiments shown that are within the scope of the claims. That is, no limitations with respect to the embodiments shown and described herein are intended or should be inferred.

Given the discussion herein (reference characters refer to the drawings discussed below), it will be appreciated that in one aspect, an exemplary method includes the operations of selecting a set of candidate drugs based on one or more outcomes related to one or more diseases (operation 254); obtaining variants linked with the selected set of candidate drugs (operation 258); collating one or more protein sequences related to the selected set of candidate drugs (operation 262); generating pairs of variants and protein sequences for each drug of the set of candidate drugs (operation 266); training a contrastive learning model 246 using the generated pairs of variants and protein sequences (operation 270); and performing a downstream task using the contrastive learning model 246 (operation 282). The technical benefits include systems and techniques for the discovery of multi-associations between proteomics, SNPs and drug interaction, and the embedding of such information for downstream analysis (e.g., foundation models (FM)); contextualization of the associations; more accurate contrastive learning models for variants and protein sequences; support for a variety of use cases, including data-driven features selections via contrastively learned embeddings and the generation of interpretable multi-omics features for downstream therapeutics and drug design tasks (such as drug repurposing); pre-training for foundation models (and other AI tasks) for drug design based on the discovered associations; systems and techniques for using proteomics, omics and drug integration with AI approaches to therapeutic discovery; systems and techniques applicable to any multi-modal data involving multi-omics categorial, and continuous variables with ranking of features for downstream candidate selection using contrastive learning-based methods; systems and techniques for building a ranking of features for the downstream selection of therapeutic candidates in healthcare data; support for downstream tasks, including discovering novel targets, the repurposing of drugs, making drugs personalized for different populations and the like; and enabling the discovery of genetic variants and their annotation in the light of their association and impact on proteins.

In one example embodiment, associations between the variants and the protein sequences are learned based on the contrastive learning model 246 (operation 274). The technical benefits include embodiments that discover many-to-many associations using drug mediation for artificial intelligence (AI) and machine learning (ML) downstream tasks and learning associations between variants and protein sequences by analyzing the parameters (such as weights) of the contrastive learning model.

In one example embodiment, a protein function is interpreted and a relationship between the protein function and the variants is determined (operation 278). The technical benefits include enhancing the efficiency of a human subject matter expert in understanding the relationship between the protein function and the variants.

In one example embodiment, the performing of the downstream task further comprises searching for novel therapeutic targets. The technical benefits include identifying novel therapeutic targets.

In one example embodiment, the performing of the downstream task further comprises identifying a disease for targeting by repurposing of a drug of the set of drugs. The technical benefits include the repurposing of drugs to treat additional diseases.

In one example embodiment, the performing of the downstream task further comprises identifying a demographic that is effectively treated by a drug of the set of drugs. The technical benefits include identifying new and/or specific demographics that are effectively treated by a drug.

In one example embodiment, embeddings for the contrastive learning model are generated based on variants. The technical benefits include capturing multiple features of variants, such as the SNP 224.

In one example embodiment, embeddings for the contrastive learning model are generated based on protein sequences. The technical benefits include capturing multiple features of the protein sequences 232.

In one example embodiment, a patient is treated based on the performance of the downstream task that uses the contrastive learning model. The technical benefits include developing prescriptions for treating a patient.

In one example embodiment, pharmaceutical equipment is controlled to synthesize a drug in accordance with results of the search for novel therapeutic targets. The technical benefits include designing and manufacturing new drugs. In example embodiments, the end user device 103 is implemented using computer-controlled chemical processing equipment which is given instructions via the LAN/WAN 102.

In one aspect, a computer program product comprises one or more tangible computer-readable storage media and program instructions stored on at least one of the one or more tangible computer-readable storage media, the program instructions executable by a processor, the program instructions comprising selecting a set of candidate drugs based on one or more outcomes related to one or more diseases (operation 254); obtaining variants linked with the selected set of candidate drugs (operation 258); collating one or more protein sequences related to the selected set of candidate drugs (operation 262); generating pairs of variants and protein sequences for each drug of the set of candidate drugs (operation 266); training a contrastive learning model 246 using the generated pairs of variants and protein sequences (operation 270); and performing a downstream task using the contrastive learning model 246 (operation 282). The technical benefits include systems and techniques for the discovery of multi-associations between proteomics, SNPs and drug interaction, and the embedding of such information for downstream analysis (e.g., foundation models (FM)); contextualization of the associations; more accurate contrastive learning models for variants and protein sequences; support for a variety of use cases, including data-driven features selections via contrastively learned embeddings and the generation of interpretable multi-omics features for downstream therapeutics and drug design tasks (such as drug repurposing); pre-training for foundation models (and other AI tasks) for drug design based on the discovered associations; systems and techniques for using proteomics, omics and drug integration with AI approaches to therapeutic discovery; systems and techniques applicable to any multi-modal data involving multi-omics categorial, and continuous variables with ranking of features for downstream candidate selection using contrastive learning-based methods; systems and techniques for building a ranking of features for the downstream selection of therapeutic candidates in healthcare data; support for downstream tasks, including discovering novel targets, the repurposing of drugs, making drugs personalized for different populations and the like; and enabling the discovery of genetic variants and their annotation in the light of their association and impact on proteins.

In one aspect, a system comprises a memory and at least one processor, coupled to the memory, and operative to perform operations comprising selecting a set of candidate drugs based on one or more outcomes related to one or more diseases (operation 254); obtaining variants linked with the selected set of candidate drugs (operation 258); collating one or more protein sequences related to the selected set of candidate drugs (operation 262); generating pairs of variants and protein sequences for each drug of the set of candidate drugs (operation 266); training a contrastive learning model 246 using the generated pairs of variants and protein sequences (operation 270); and performing a downstream task using the contrastive learning model 246 (operation 282). The technical benefits include systems and techniques for the discovery of multi-associations between proteomics, SNPs and drug interaction, and the embedding of such information for downstream analysis (e.g., foundation models (FM)); contextualization of the associations; more accurate contrastive learning models for variants and protein sequences; support for a variety of use cases, including data-driven features selections via contrastively learned embeddings and the generation of interpretable multi-omics features for downstream therapeutics and drug design tasks (such as drug repurposing); pre-training for foundation models (and other AI tasks) for drug design based on the discovered associations; systems and techniques for using proteomics, omics and drug integration with AI approaches to therapeutic discovery; systems and techniques applicable to any multi-modal data involving multi-omics categorial, and continuous variables with ranking of features for downstream candidate selection using contrastive learning-based methods; systems and techniques for building a ranking of features for the downstream selection of therapeutic candidates in healthcare data; support for downstream tasks, including discovering novel targets, the repurposing of drugs, making drugs personalized for different populations and the like; and enabling the discovery of genetic variants and their annotation in the light of their association and impact on proteins.

In one aspect, a system comprises a first transformer 228 configured to generate embeddings for a contrastive learning model based on variants, the variants linked with a selected set of candidate drugs, the set of candidate drugs selected based on one or more outcomes related to one or more diseases; a second transformer 236 configured to generate embeddings for the contrastive learning model based on protein sequences; a contrastive learning model 246 trained using the variant-based embeddings, the protein sequences-based embeddings and generated pairs of the variants and the protein sequences; and a software component configured to implement a downstream task using the contrastive learning model. The technical benefits include systems and techniques for the discovery of multi-associations between proteomics, SNPs and drug interaction, and the embedding of such information for downstream analysis (e.g., foundation models (FM)); contextualization of the associations; more accurate contrastive learning models for variants and protein sequences; support for a variety of use cases, including data-driven features selections via contrastively learned embeddings and the generation of interpretable multi-omics features for downstream therapeutics and drug design tasks (such as drug repurposing); pre-training for foundation models (and other AI tasks) for drug design based on the discovered associations; systems and techniques for using proteomics, omics and drug integration with AI approaches to therapeutic discovery; systems and techniques applicable to any multi-modal data involving multi-omics categorial, and continuous variables with ranking of features for downstream candidate selection using contrastive learning-based methods; systems and techniques for building a ranking of features for the downstream selection of therapeutic candidates in healthcare data; support for downstream tasks, including discovering novel targets, the repurposing of drugs, making drugs personalized for different populations and the like; and enabling the discovery of genetic variants and their annotation in the light of their association and impact on proteins.

Generally, an exemplary contrastive learning-based framework, referred to as PGxProt herein, is disclosed (references to PGxProt are to be understood as references to an exemplary embodiment and the description in the specification of certain features with respect to PGxProt are not intended to be read into the claims unless expressly recited therein). In one or more embodiments, the contrastive learning-based framework trains a foundation model to find the associations between pharmacogenomic (PGx) induced variants and proteins and to thereby relate protein functions to genomic markers. Exemplary embodiments of the pharmacogenomic induced protein function method (PGxProt) can be used to directly compute:
a transfer-based multi-omics method to integrate genomics and proteomics markers mediated by therapeutic candidates of complex diseases; and
interpretable associations between protein functions and genomic markers for effective drug discovery.

Exemplary embodiments of the pharmacogenomic (PGx) induced protein function method (PGxProt) provides for the efficient selection of candidates of therapeutics discovery for complex diseases and generates pairs of PGx markers and proteins related to their underlying biological function. The association of SNPs with the function of proteins (including antibodies of therapeutic candidates) is beneficial for a variety of tasks, including:
furthering the understanding of precision medicine candidates;
development of more targeted therapeutic candidates;
reduced adverse drug reactions; and
enabling the discovery of drug repurposing candidates.

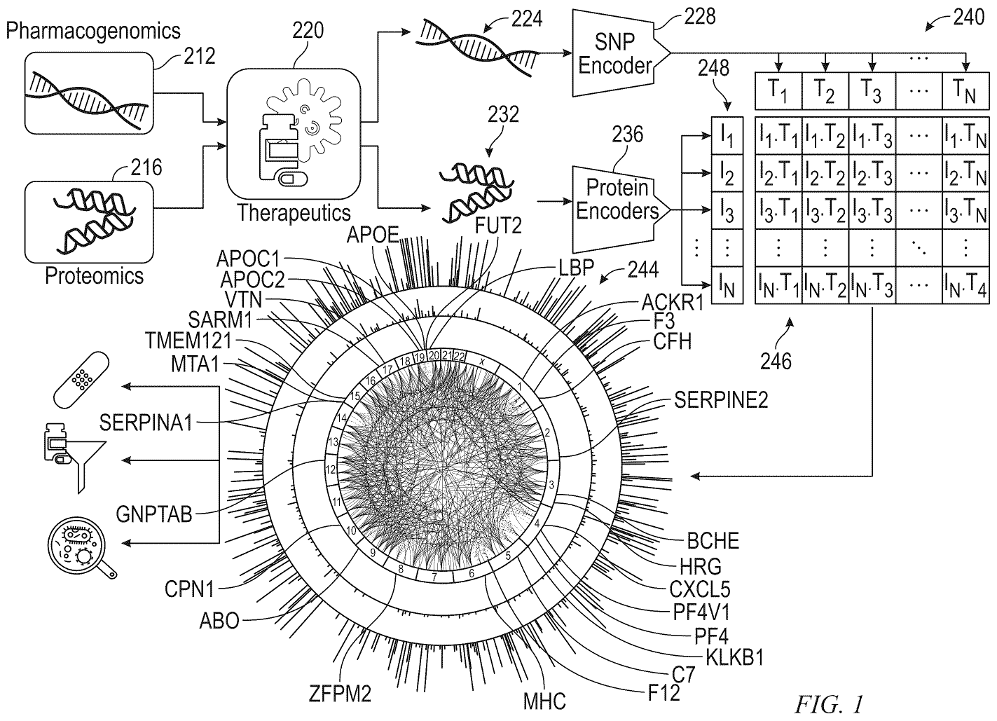

FIG. 1 is a high-level workflow for learning associations between proteins and variants (such as SNPs), in accordance with an example embodiment. In one example embodiment, information on medications is analyzed to identify relationships between SNPs and proteins, where the SNPs express pathogenic mutants. In one example embodiment, contrastive learning is used between the protein function and pharmacogenomics (PGx) induced variants, and computes transfer-based multi-omics methods to integrate genomics and proteomics markers mediated by therapeutic candidates of complex diseases. (Contrastive learning is a machine learning technique that trains a model to distinguish between pairs of similar and dissimilar instances-given the teachings herein, the skilled artisan can implement suitable machine learning techniques in software on a general purpose computer (optionally with a hardware accelerator); software on a special-purpose computer such as an array of graphics processing units (GPUs); non-Von Neumann machines; hardware; or a mixture of the forgoing.) The SNP and protein information can be stored in tabular format with discrete variables for the former corresponding to dosages of each mutation and quantitative values corresponding to expression for the proteins. One or more embodiments produce interpretable associations between protein functions and genomic markers for effective drug discovery. Pairs of PGx markers 212 and proteins 216 showing the relationship of SNPs 224 and proteins 232 related to their underlying biological function are created based on therapeutic information 220. Each pair of SNP 224 protein 232 and protein 232 is encoded by transformers 228, 236, respectively, to generate SNP embeddings 240 and protein embeddings 248, respectively. (A transformer is a type of neural network architecture that transforms an input sequence into an output sequence-given the teachings herein, the skilled artisan can implement suitable neural networks in software on a general purpose computer (optionally with a hardware accelerator); software on a special-purpose computer such as an array of graphics processing units (GPUs); non-Von Neumann machines; hardware; or a mixture of the forgoing.) The resulting SNP embeddings 240 and protein embeddings 248 are used to train the model 246. In one example embodiment, the model 246 is a contrastive learning model trained to relate SNPs 224 with proteins 232, based on relationships known in the context of certain drugs.

In one example embodiment, medications where the drugs are known to target certain proteins, such as statins, blood thinners and the like, are evaluated. These medications are also often linked with certain GGx markers (SNPs); thus, protein/SNP pairs can be identified and created based on the information associated with the medications. In some cases, a drug can lead to the generation of hundreds or thousands of SNP-protein pairs. Once the relevant protein/SNP pairs are identified, a first transformer model is used to encode proteins into protein encoded embeddings and a second transformer model is used to encode the SNPs into SNP encoded embeddings. A contrastive learning framework learns a loss function that takes into account both encodings and outputs a weight for each pair (the generated weights can be formatted in a matrix based on the corresponding SNP and protein, and records a SNP-protein pair of the association that have been validated with respect to and in the context of the other relationships between SNPs and proteins (such as how the SNP and the protein interact as this can be instrumental in confirming the validity of the relationship). Conventional techniques, such as logistic regression models, do not consider the context of other relationships.

In one example embodiment, a circle plot 244 is generated based on the learned model 246 and the protein/SNP pairs. The circle plot is computed using the effect sizes (the weights obtained from the model that correspond to an identified association) of associations between proteins and SNPs for statistically significant tests with p-value less than 0.05. The effect sizes can be obtained using multiple association testing or from the FM attention matrix. The circle plot 244 and matrix of weights assists in developing a user dashboard for repurposing drugs; discovering SNPs for targeting new diseases; determining, for example, the SNPs associated with a given a protein; determining, if a drug is related to a protein, the related SNPs of the drug and protein, and determining other drugs that are related to the protein; and the like. A fine-grained analysis of the biological underpinning of a certain drug can be discovered, which can reduce misclassification of drugs (such as the misclassification of drugs across different demographics, since each SNP is typically related to particular demographics).

### Computing Associations Between PGx Induced Variants and Proteins

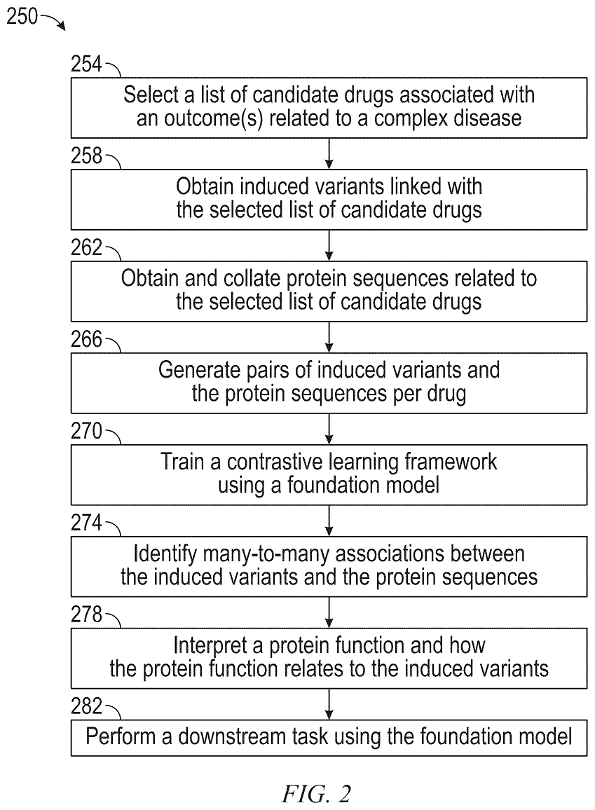

FIG. 2 is a flowchart 250 for an example method for learning associations between proteins and SNPs, in accordance with an example embodiment. The input includes, for example, a list of drugs, a PGx repository and a protein data bank. The output includes, for example, many-to-many associations between PGx induced variants and proteins. In one example embodiment, a list of candidate drugs associated with an outcome or outcomes related to a given complex disease is selected (operation 254). The selection of candidate drugs is a design choice. For example, drugs associated with cardiovascular disease may be selected and can lead to the identification of 5,000, 50,000 or more pairs. The PGx variants linked with the selected drugs from operation 254 are obtained from a conventional PGx repository (operation 258). The protein sequences related to the selected drugs from operation 204 are obtained from the protein data bank and collated (operation 262). For example, the proteins may be collated by their coordinates as obtained from the protein data bank. Pairs of induced variants and protein sequences for each drug in the list of candidate drugs are created (operation 266). In one example embodiment, the PGx repository is queried and analyzed to identify proteins and induced variants, such as SNPs, which are associated with each drug in the list of candidate drugs to create the pairs. The model 246 of the contrastive learning framework is trained using a foundation model (operation 270). For example, each pair of protein and SNP is encoded by transformers 228, 236, respectively. The resulting protein embeddings 248 and SNP embeddings 240 are used to train the model 246.

Many-to-many associations between the PGx induced variants and the protein sequences are identified (operation 274). In one example embodiment, the weights of the learned model 246 are queried and the pairs corresponding to the largest weights are identified as having relevant relationships between the protein and the corresponding SNP. In one example embodiment, the weights are sorted and the weights that lie in the higher quantile (such as a decile) of the distribution of the weights are selected.

A protein function, and how the protein function relates to the PGx variants, are interpreted (operation 278). In one example embodiment, relationships of proteins, SNPs and diseases are interpreted by understanding the corresponding biological functions and by comparing discovered weights and ground truths. As an example, a known SNP target in a coding region will be associated with a protein target with higher weight than a SNP in the non-coding region with respect to that protein target. This can be validated by external databases with protein-SNP associations and in the literature. A circle plot 244, as described above, is also generated. Other example interpretations include determining how a protein that relates to drug A also relates to drug B, determining which demographics a SNP is associated with, and the like. The circle plot enables an investigation of the regions of proteins and SNPs which are most associated and determining if there are non-coding SNPs which are associated with proteins, which, in turn will enable the discovery of novel therapeutic targets.

In one example embodiment, downstream tasks are performed using the trained model 246 (operation 282). For example, if a protein that is suspected of providing a therapeutic is identified, and the related proteins and SNPs are known, the generated model 246 can be used to infer weights for these new pairs of protein and induced PGx variants. As described above, examples of downstream tasks include discovering novel targets (proteins), the repurposing of drugs, making drugs personalized for different populations, and the like.

In one example embodiment, a given medication that targets a given disease is repurposed to target another disease by recognizing that at least some of the proteins and SNPs associated with the given medication are also associated with the other disease. In one example embodiment, responsive to the repurposing analysis, the repurposed medication is administered to a patient to treat the other disease.

One or more exemplary embodiments discover SNPs for targeting new diseases by finding SNPs associated with known proteins for certain diseases.

In one example embodiment, the SNPs associated with a given a protein are determined based on the pairs created in operation 266.

In one example embodiment, if a first drug is related to a protein, other drugs that are related to the protein are identified and considered as a candidate for treating a disease targeted by the first drug.

In one example embodiment, a fine-grained analysis of the biological underpinning of a specified drug is generated and used to reduce a misclassification of drugs (such as a misclassification of drugs across different demographics resulting from each demographic being related to different SNPs). The fine-grained analysis involves identifying underlying biological pathways of the SNP and obtaining their frequencies in different populations across the world to quantify misclassification (as would be appreciated by the skilled artisan given the teachings herein, the fine-grained analysis can be carried out identifying underlying biological pathways of the SNP).

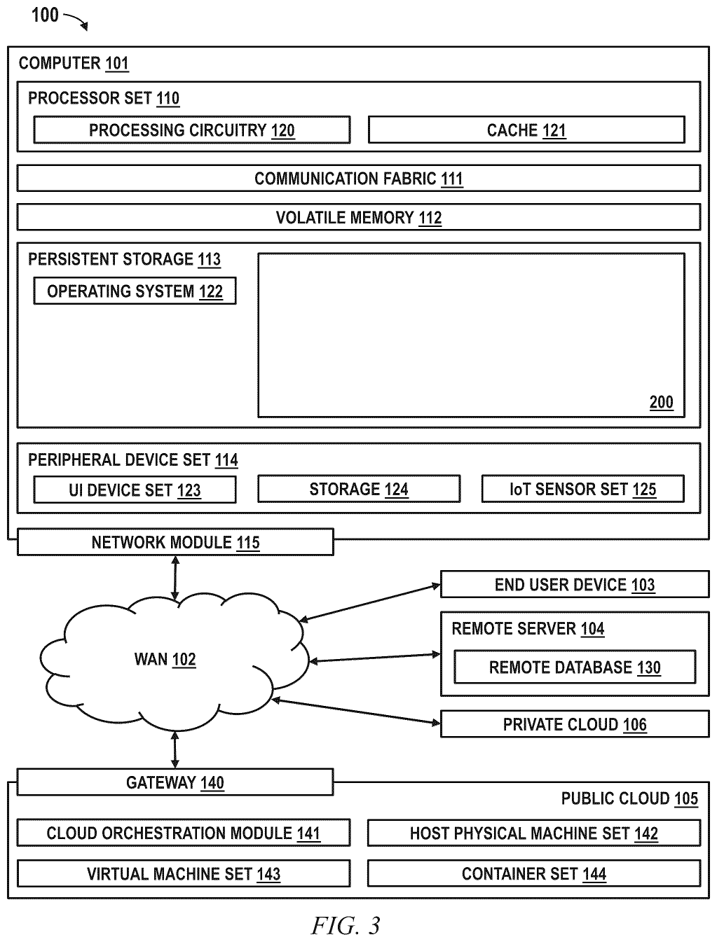

Refer now to FIG. 3.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

Computing environment 100 contains an example of an environment for the execution of at least some of the computer code involved in performing the inventive methods, such as pharmacogenomic protein association system 200. In addition to block 200, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and block 200, as identified above), peripheral device set 114 (including user interface (UI) device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144. In example embodiments, the pharmacogenomic system 200 interfaces with end user device 103 (such as pharmaceutical synthesis equipment) via WAN 102, to control synthesis of identified pharmaceuticals.

COMPUTER 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 3. On the other hand, computer 101 is not required to be in a cloud except to any extent as may be affirmatively indicated.

PROCESSOR SET 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in block 200 in persistent storage 113.

COMMUNICATION FABRIC 111 is the signal conduction path that allows the various components of computer 101 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

VOLATILE MEMORY 112 is any type of volatile memory now known or to be developed in the future. Examples include dynamic type random access memory (RAM) or static type RAM. Typically, volatile memory 112 is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

PERSISTENT STORAGE 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid state storage devices. Operating system 122 may take several forms, such as various known proprietary operating systems or open source Portable Operating System Interface-type operating systems that employ a kernel. The code included in block 200 typically includes at least some of the computer code involved in performing the inventive methods.

PERIPHERAL DEVICE SET 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion-type connections (for example, secure digital (SD) card), connections made through local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

NETWORK MODULE 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN 102 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 103 is any computer system that is used and controlled by an end user (for example, a customer of an enterprise that operates computer 101), and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

REMOTE SERVER 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

PUBLIC CLOUD 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A computer-implemented method comprising:
selecting a set of candidate drugs based on one or more outcomes related to one or more diseases;
obtaining variants linked with the selected set of candidate drugs;
collating one or more protein sequences related to the selected set of candidate drugs;
generating pairs of variants and protein sequences for each drug of the set of candidate drugs;
training a contrastive learning model using the generated pairs of variants and protein sequences; and
performing a downstream task using the contrastive learning model.

2. The computer-implemented method of claim 1, further comprising learning associations between the variants and the protein sequences based on the contrastive learning model.

3. The computer-implemented method of claim 1, further comprising interpreting a protein function and determining a relationship between the protein function and the variants.

4. The computer-implemented method of claim 1, wherein the performing the downstream task further comprises searching for novel therapeutic targets.

5. The computer-implemented method of claim 1, wherein the performing the downstream task further comprises identifying a disease for targeting by repurposing of a drug of the set of drugs.

6. The computer-implemented method of claim 1, wherein the performing the downstream task further comprises identifying a demographic that is effectively treated by a drug of the set of drugs.

7. The computer-implemented method of claim 1, generating embeddings for the contrastive learning model based on variants.

8. The computer-implemented method of claim 1, generating embeddings for the contrastive learning model based on protein sequences.

9. The computer-implemented method of claim 1, treating a patient based on the performance of the downstream task that uses the contrastive learning model.

10. The computer-implemented method of claim 1, controlling pharmaceutical equipment to synthesize a drug in accordance with results of the search for novel therapeutic targets.

11. A computer program product, comprising:
one or more tangible computer-readable storage media and program instructions stored on at least one of the one or more tangible computer-readable storage media, the program instructions executable by a processor, the program instructions comprising:
selecting a set of candidate drugs based on one or more outcomes related to one or more diseases;
obtaining variants linked with the selected set of candidate drugs;
collating one or more protein sequences related to the selected set of candidate drugs;
generating pairs of variants and protein sequences for each drug of the set of candidate drugs;
training a contrastive learning model using the generated pairs of variants and protein sequences; and
performing a downstream task using the contrastive learning model.

12. The computer program product of claim 11, the program instructions further comprising learning associations between the variants and the protein sequences based on the contrastive learning model.

13. The computer program product of claim 11, the program instructions further comprising interpreting a protein function and determining a relationship between the protein function and the variants.

14. The computer program product of claim 11, wherein the performing the downstream task further comprises searching for novel therapeutic targets.

15. The computer program product of claim 11, wherein the performing the downstream task further comprises identifying a disease for targeting by repurposing of a drugs of the set of drugs.

16. The computer program product of claim 11, wherein the performing the downstream task further comprises identifying a demographic that is effectively treated by a drug of the set of drugs.

17. The computer program product of claim 11, the program instructions further comprising generating embeddings for the contrastive learning model based on variants.

18. The computer program product of claim 11, the program instructions further comprising generating embeddings for the contrastive learning model based on protein sequences.

19. A system comprising:
a memory; and
at least one processor, coupled to said memory, and operative to perform operations comprising:
selecting a set of candidate drugs based on one or more outcomes related to one or more diseases;
obtaining variants linked with the selected set of candidate drugs;
collating one or more protein sequences related to the selected set of candidate drugs;
generating pairs of variants and protein sequences for each drug of the set of candidate drugs;
training a contrastive learning model using the generated pairs of variants and protein sequences; and
performing a downstream task using the contrastive learning model.

20. The system of claim 19, the operations further comprising learning associations between the variants and the protein sequences based on the contrastive learning model.

21. The system of claim 19, the operations further comprising interpreting a protein function and determining a relationship between the protein function and the variants.

22. The system of claim 19, wherein the performing the downstream task further comprises searching for novel therapeutic targets.

23. The system of claim 19, wherein the performing the downstream task further comprises identifying a disease for targeting by repurposing of a drug of the set of drugs.

24. The system of claim 19, wherein the performing the downstream task further comprises identifying a demographic that is effectively treated by a drug of the set of drugs.

25. A system comprising:
a first transformer configured to generate embeddings for a contrastive learning model based on variants, the variants linked with a selected set of candidate drugs, the set of candidate drugs selected based on one or more outcomes related to one or more diseases;
a second transformer configured to generate embeddings for the contrastive learning model based on protein sequences;
a contrastive learning model trained using the variant-based embeddings, the protein sequences-based embeddings and generated pairs of the variants and the protein sequences; and
a software component configured to implement a downstream task using the contrastive learning model.
